(19) United States
(12) Patent Application Publication
(10) Pub. No.: US 2024/0091289 A1
(43) Pub. Date: Mar. 21, 2024

(54) Lichen Extract-based Formulation for Cervids

(71) Applicant: Zoo Pharma Dynamics, LLC, Hondo, TX (US)

(72) Inventor: Brian Kanke, Hondo, TX (US)

(21) Appl. No.: 18/030,374

(22) PCT Filed: Jul. 27, 2021
(86) PCT No.: PCT/US21/70996

§ 371 Date: Apr. 5, 2023

Related U.S. Application Data
(60) Provisional application No. 63/056,793, filed on Jul. 27, 2020.

(51) Int. Cl. A61K 36/09; A01N 25/04; A01N 59/02; A01N 65/00; A23K 10/30; A23K 50/10; A61K 9/00; C11D 3/38; C11D 3/43; C11D 3/48; C11D 17/00

(52) U.S. Cl. CPC A61K 36/09; A01N 25/04; A01N 59/02; A01N 65/00; A23K 10/30; A23K 50/10; A61K 9/0095; C11D 3/381; C11D 3/43; C11D 3/48; C11D 17/0043

(57) ABSTRACT

A lichen extract-based formulation containing a mixture of reindeer lichen extract (Cladonia rangiferina), lung lichen extract (Lobaria pulmonaria), and witch's hair lichen extract (Alectoria sarmentosa); feed pellet, protein block and solution containing the lichen extract-based formulation; lichen extract-based spray cleaner formulation and methods for administration of the formulations to cervids are disclosed.

This application claims the benefit of the filing date of U.S. Provisional Patent Application Ser. No. 63/056,793, filed Jul. 27, 2020, which is hereby incorporated by reference in its entirety.

### FIELD

The present disclosure relates to a lichen extract-based formulation; feed pellet, protein block and solution containing the base formulation; lichen extract-based spray cleaner and methods for administration of the formulations to cervids.

### BACKGROUND

Chronic wasting disease (CWD) is a prion-related disease that effects cervids, such as deer, elk, reindeer, sika deer, and moose. CWD is problematic as more animals come into contact with infectious prions, by direct contact with an infected cervid or viable CWD prions in the environment. Transmission among cervids is enhanced by activities that promote the congregation of animals, such as baiting or feeding. As more cervids become infected, the subsequent environmental contamination multiplies, playing a key role in the continued spread of CWD.

Prior research (Prion Research Laboratory, 2002) has suggested that some lichens (extracts from Lobaria pulmonaria and Cladonia rangiferina) on the landscape could have potential to break down the infectious prions that are responsible for causing chronic wasting disease. Lichens produce unique organic compounds that have antibiotic and antiviral properties.

### SUMMARY

In accordance with one aspect of the present disclosure, there is provided a cervid base formulation including a mixture of reindeer lichen extract (Cladonia rangiferina), lung lichen extract (Lobaria pulmonaria), and witch's hair lichen extract (Alectoria sarmentosa).

In accordance with another aspect of the present disclosure, there is provided a formulation containing a lichen extract mixture; protein; fat; fiber; vitamins; minerals; a preservative and an emulsifier, in the form of a protein block, wherein the mixture contains from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

In accordance with another aspect of the present disclosure, there is provided a formulation containing a lichen extract mixture; protein; fat; fiber; vitamins; minerals; a preservative and an emulsifier, in the form of a feed supplement, wherein the mixture comprises from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

In accordance with another aspect of the present disclosure, there is provided a lichen extract-based spray cleaner formulation, including: an ingredient of at least one of reindeer lichen extract (Cladonia rangiferina) and lung lichen extract (Lobaria pulmonaria); an enzyme stabilizer; preservative; antimicrobial active; and a carrier solvent.

In accordance with another aspect of the present disclosure, there is provided a method, including administering to cervids a composition comprising a mixture of reindeer lichen extract (Cladonia rangiferina), lung lichen extract (Lobaria pulmonaria), and witch's hair lichen extract (Alectoria sarmentosa).

These and other aspects of the present disclosure will become apparent upon a review of the following detailed description and the claims appended thereto.

### BRIEF DESCRIPTION OF THE DRAWINGS

FIG. 1 is a photograph of a protein block (PPB) in accordance with the present disclosure;

FIG. 2 is a photograph of a feed supplement (PPFS) containing base formulation (CPP) in accordance with the present disclosure;

FIG. 3 is a photograph of a powdered base formulation (PWP) containing base formulation (CPP) for use in water in accordance with the present disclosure;

FIG. 4 is a photograph of bottles of spray cleaner that contain various formulations in accordance with the present disclosure;

FIG. 5 is a photograph of the 5% Lung Lichen Formulation before and after the stability chamber;

FIG. 6 is a photograph of the 10% Lung Lichen Formulation before and after the stability chamber;

FIG. 7 is a photograph of the 15% Lung Lichen Formulation before and after the stability chamber;

FIG. 8 is a photograph of the 20% Lung Lichen Formulation before and after the stability chamber;

FIG. 9 is a photograph of the 5% Reindeer Lichen Formulation before and after the stability chamber;

FIG. 10 is a photograph of the 10% Reindeer Lichen Formulation before and after the stability chamber;

FIG. 11 is a photograph of the 15% Reindeer Lichen Formulation before and after the stability chamber; and

FIG. 12 is a photograph of the 20% Reindeer Lichen Formulation before and after the stability chamber.

### DETAILED DESCRIPTION

The present disclosure relates to various formulations and methods for administration to cervids. The cervid base formulation is an additive for use in pelleted feeds, protein blocks, drinking water and in a spray cleaner for use on cervid's and certain livestock facilities. The cervid base formulation is a combination of 3 plant extracts. The cervid base formulation includes extracts of Reindeer Lichen (Cladonia rangiferina), Lung Lichen (Lobaria pulmonaria) and Witch's Hair Lichen (Alectoria sarmentosa). Formulations containing the Witch's Hair Lichen (Alectoria sarmentosa) extract are 75% more digestible than formulations without this extract.

The whole plants are harvested and processed into a powder form through high-temperature sterilization. They appear as a fine brown yellow powder with a moderate odor and a moderate taste. The powder is tested for heavy metals and conforms to standards. The powder undergoes microbial tests and conforms to standards.

Through the process of high-temperature sterilization the whole plants are extracted in extract ratios of 10:1, 20:1, 30:1 and 40:1. Exact extract ratios are used for specific product formulation.

The Reindeer Lichen Extract (Cladonia rangiferina) is used at a percentage range of from 1% to 40% by weight as an additive to the specific product being manufactured.

The Lung Lichen Extract (Lobaria pulmonaria) is used at a percentage range of from 1% to 40% by weight as an additive to specific product being manufactured.

Witch's Hair Lichen Extract (Alectoria sarmentosa) are used at a percentage range of 5% to 45% by weight as an additive to specific product being manufactured.

Preferred base formulations contain extract ratios averaging 10:1, with a base percentage of each product used at an average of 15%.

In an embodiment, the base formulation contains certain preservatives and emulsifiers in the manufacturing process for stability. The base formulation further contains certain minerals used as carriers in the system of the animal. Suitable additives include plant protein products, grain products, monocalcium phosphate, molasses products, calcium carbonate, dicalcium phosphate, salt, manganous oxide, ferrous sulfate, iron oxide, zinc oxide, copper sulfate, cobalt carbonate, ethylendiamine duhyriodide, vitamin A supplement, vitamin D3 supplement, natural and artificial colors, vegetable oil, sodium selenite, processed grain by-products, maganesium-mica, potassium chloride, calcium iodate, vitamin E supplement, zinc sulfate, manganese sulfate, mineral oil, cobalt carbonate, diatomaceous earth, dried seaweed meal, chickory root, cloves, saccharin sodium, hemicellulose extract, origanum oil, lemongrass oil, processed grain by products, forage products, Saccharomyces cerevisae, dried Bacillus licheniformis extract, vitamin E supplement, vitamin B12 supplement, d-biotin, riboflavin supplement, d-calcium pantothenate, menadione sodium bisulfate complex, folic acid, thiamine mononitrate, choline chloride, zinc amino acid complex, manganese amino acid complex, copper amino acid complex, cobalt sulfate, cobalt glucoheptonate, lichen extracts and artificial flavors.

The cervid feed supplement is a pelleted cervid feed product containing the base formulation, which is a mixture of reindeer lichen extract (Cladonia rangiferina), lung lichen extract (Lobaria pulmonaria), and witch's hair lichen extract (Alectoria sarmentosa). In an embodiment, the feed supplement contains protein; fat; fiber; vitamins; minerals; a preservative and an emulsifier, in the form of a feed supplement, wherein the mixture contains from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

The cervid protein block product contains the base formulation, which is a mixture of reindeer lichen extract (Cladonia rangiferina), lung lichen extract (Lobaria pulmonaria), and witch's hair lichen extract (Alectoria sarmentosa). In an embodiment, the protein block contains protein; fat; fiber; vitamins; minerals; a preservative and an emulsifier, in the form of a protein block, wherein the mixture contains from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

The cervid water solution contains the base formulation, which is a mixture of reindeer lichen extract (Cladonia rangiferina), lung lichen extract (Lobaria pulmonaria), and witch's hair lichen extract (Alectoria sarmentosa). 100% of the base formulation can be mixed with water and the solution can be added to the water source of cervids.

The spray product is used as a spray cleaner in livestock facilities to include feed troughs, water tubs, and the like. This spray cleaner product contains a lichen extract. Preferably, the lichen extract is present in an amount of from 5% to 40%. A suitable lichen extract contains reindeer lichen extract (Cladonia rangiferina), lung lichen extract (Lobaria pulmonaria) or a mixture thereof, or the base formulation, which is a mixture of reindeer lichen extract (Cladonia rangiferina), lung lichen extract (Lobaria pulmonaria), and witch's hair lichen extract (Alectoria sarmentosa.

The disclosure will be further illustrated with reference to the following specific examples. It is understood that these examples are given by way of illustration and are not meant to limit the disclosure or the claims to follow.

### Example 1—Protein Block

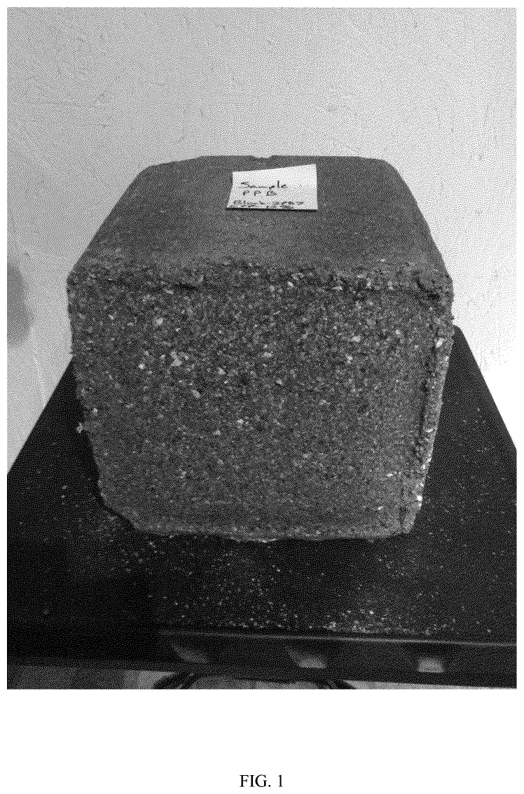

This example sets forth a formulation in the form of a protein block shown in FIG. 1 in accordance with the present disclosure:

| | | |
|---|---|---|
| Crude Protein | 20.00% | min |
| Crude Fat | 2.0% | min |
| Crude Fiber | 8.0% | max |
| Calcium | 5.0% | min |
| Calcium | 10.0% | max |
| Phosphorus | 3.0% | min |
| Potassium | 1.0% | min |
| Salt | 1.0% | min |
| Salt | 10.0% | max |
| Copper | 500 | ppm min |
| Selenium | 2.0 | ppm min |
| Zinc | 200 | ppm min |
| Vitamin A | 18,000 | IU/lb min |
| Vitamin D3 | 2,000 | IU/lb min |
| Vitamin E | 75 | IU/lb min |
| Base Formulation | 1.0% | min |

### Ingredients:

Plant protein products, grain products, monocalcium phosphate, molasses products, calcium carbonate, dicalcium phosphate, salt, manganous oxide, ferrous sulfate, iron oxide, zinc oxide, copper sulfate, cobalt carbonate, ethylendiamine duhyriodide, vitamin A supplement, vitamin D3 supplement, natural and artificial colors, vegetable oil, sodium selenite, processed grain by-products, maganesium-mica, potassium chloride, calcium iodate, vitamin E supplement, zinc sulfate, manganese sulfate, mineral oil, cobalt carbonate, diatomaceous earth, dried seaweed meal, chickory root, cloves, saccharin sodium, hemicellulose extract, origanum oil, lemongrass oil, lichen extracts.

### Example 2—Feed Supplement

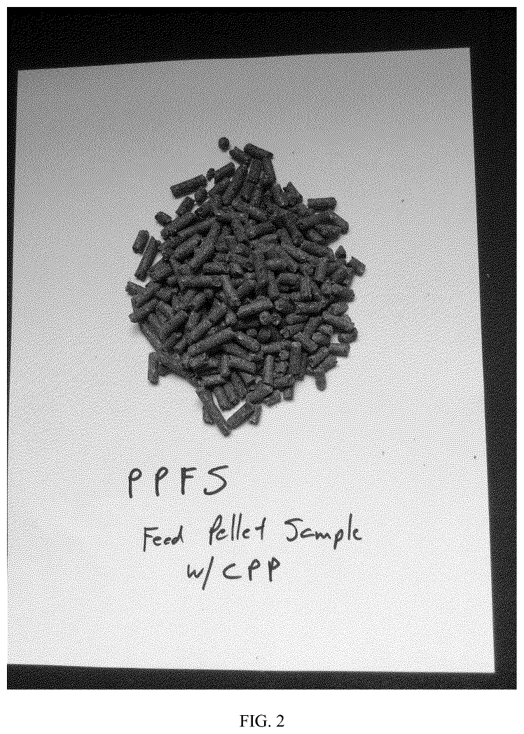

This example sets forth a formulation for a feed supplement shown in FIG. 2 in accordance with the present disclosure:

| | | |
|---|---|---|
| Crude Protein | 16.0% | min |
| Lysine | 1.0% | min |
| Crude Fat | 6.0% | min |
| Crude Fiber | 20.0% | max |
| Calcium | 5.0% | min |
| Phosphorus | 1.0% | min |
| Salt | 6.0% | max |
| Copper | 500 | ppm min |
| Manganese | 40 | ppm min |
| Selenium | 3.0 | ppm min |
| Zinc | 250 | ppm min |
| Vitamin A | 20,000 | IU/lb min |
| Vitamin D3 | 2000 | IU/lb min |
| Vitamin E | 55 | IU/lb min |
| Base Formulation | 1.0% | min |

### Ingredients:

Processed grain by products, forage products, plant protein products, can molasses, dicalcium phosphate, monocalcium phosphate, calcium carbonate, salt, Saccharomyces cerevisae, dried Bacillus licheniformis extract, vitamin A supplement, vitamin D3 supplement, vitamin E supplement, vitamin B12 supplement, d-biotin, riboflavin supplement, d-calcium pantothenate, menadione sodium bisulfate complex, folic acid, thiamine mononitrate, choline chloride, zinc sulfate, zinc amino acid complex, manganese sulfate, manganese amino acid complex, copper sulfate, copper amino acid complex, ethylenediamine dihydriodide, cobalt sulfate, cobalt glucoheptonate, sodium selenite, lichen extracts and artificial flavors.

### Example 3—Drinking Water Formulation for Cervids

### NonSterile

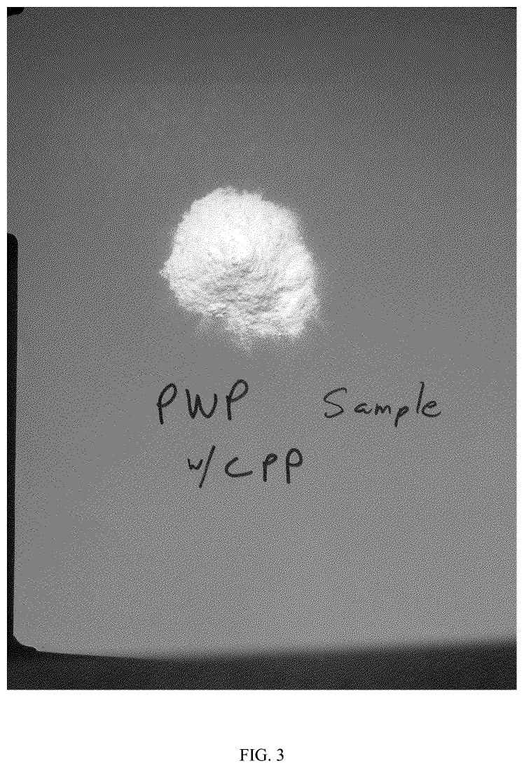

Active Ingredient: 100% Base Formulation powder shown in FIG. 3
Mix 1 packet of the Base Formulation (5 ounces of base formulation to 50 gallons of water)

### Indications:

Administration: Administer orally in cervid drinking water for 5 days every 30 days.
Directions: Combine contents of entire package in half a gallon of water in glass or plastic container. Stir to dissolve. Allow the concentrated solution to stand until foam disappears. Add solution to water source.
Residue Warnings: There are no residue warnings.

### Precautions:

For best results use a clean water source with fresh water.
NOT FOR HUMAN USE

### Example 4—Spray Product

This example tests the stability of various spray cleaner formulations.

Lichen extract-based spray cleaners were made based on the formulations below. All formulations other than the 5% Lung formulation were found to have preferred stability. The good samples demonstrated no visual difference, odor difference, or change in the ability to be sprayed after the aging process.

TABLE 2
Formulations from Previous Work

| Ingredient | Amount (Weight %) | Purpose |
|---|---|---|
| Lichen Extract | 5, 10, 15, 20% | Base Ingredient |
| (Lung/Reindeer) | | |
| Glycerol | 5, 10, 15, 20% | Enzyme Stabilizer/Solvent |
| Sodium Benzoate | 1% | Preservative |
| Sodium Bisulfate | 1% | Antimicrobial Active |
| Water | 88, 78, 68, 58% | Carrier solvent |

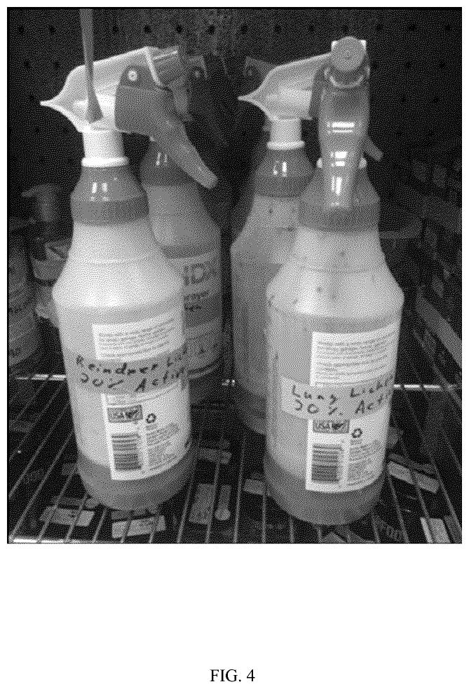

Reindeer and Lung lichen extract spray cleaners were made with 5, 10, 15, and 20% lichen extract and glycerol in accordance with Table 2. Each was made to 300 g batches. Each spray formulation was placed in its own 32-ounce spray bottle. The spray bottles were labelled and were checked to test the ability of the formulation to be sprayed onto a surface. FIG. 4 shows the spray bottles that contain the formulations. After spray testing the spray bottles were placed in a stability chamber for 7 days (40° C., 75% Humidity) to check stability. The stability chamber allows for a controlled environment regarding temperature and humidity to test the stability of the sample to set temperatures and humidity.

Approximately 20 mL of each formulation was retained before spray bottles were placed in stability chamber. After 7 days ˜20 mL of each formulation was collected for visual comparison to samples collected before stability testing. Each bottle was sprayed again to ensure the samples still sprayed easily.

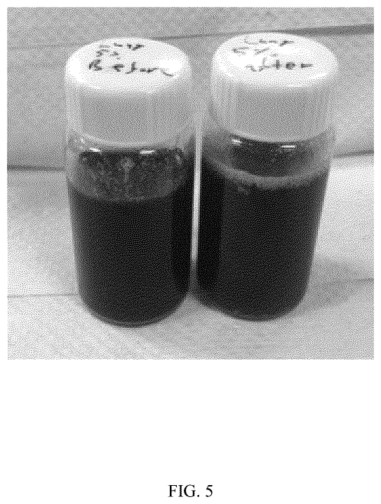

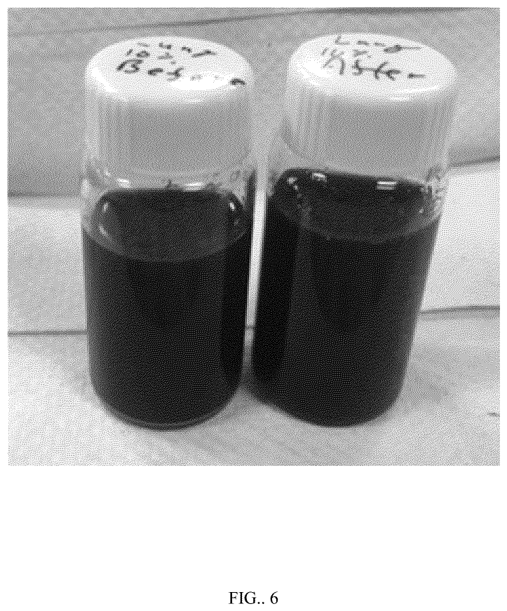

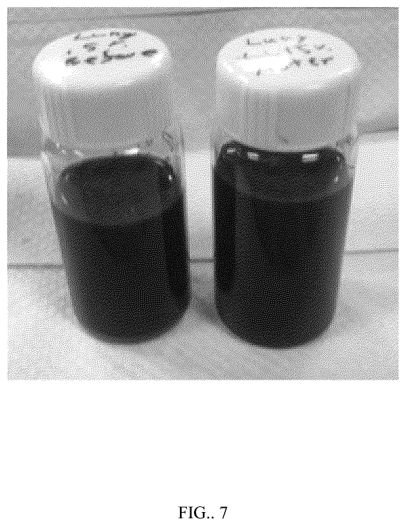

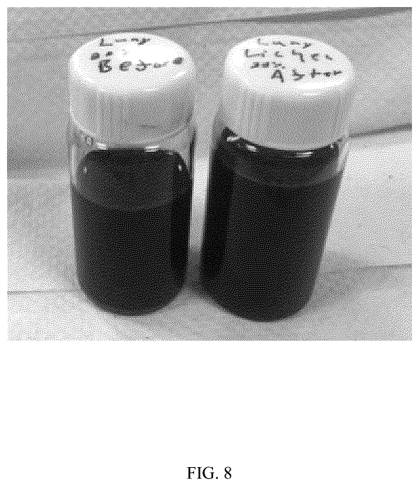

FIG. 5 (5%), FIG. 6 (10%), FIG. 7 (15%), FIG. 8 (20%) show the before and after stability testing for the Lung Lichen formulations. The 5% formulation had floating particles after the testing. This is classified as unacceptable. The 10%, 15%, 20% Lung Lichen formulations had no notable differences after testing. All formulations were able to be sprayed before and after stability testing.

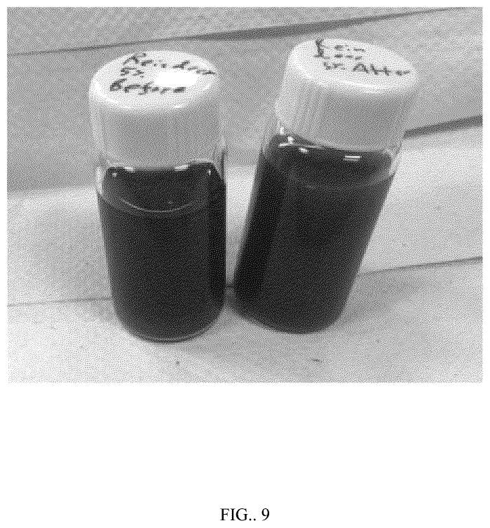

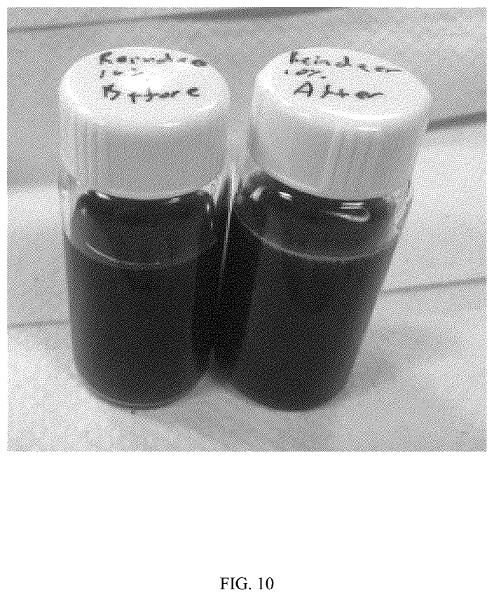

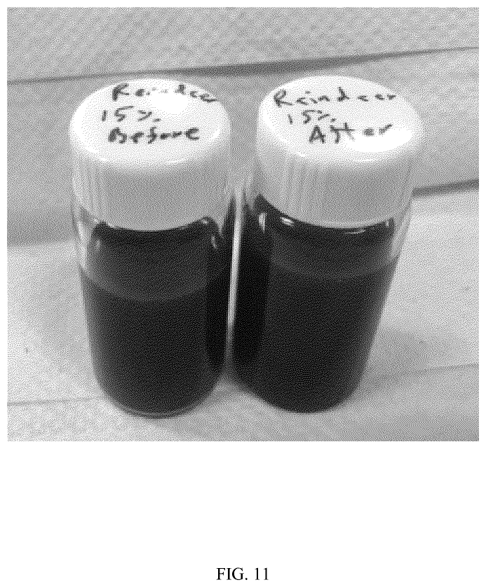

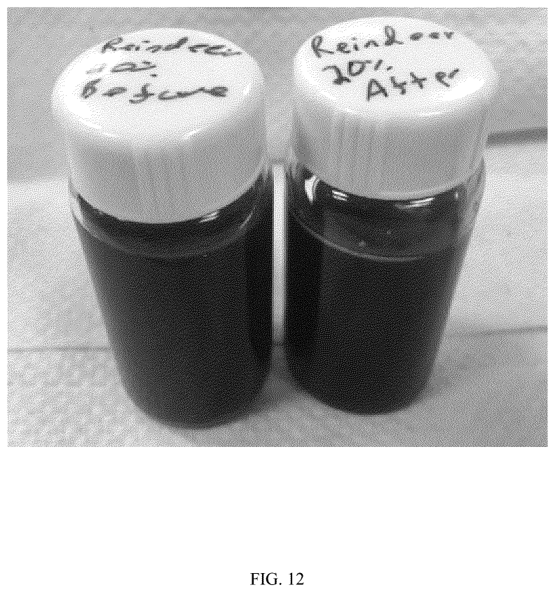

FIG. 9 (5%), FIG. 10 (10%), FIG. 11 (15%), FIG. 12 (20%) show the before and after stability testing for the Reindeer Lichen spray formulations. The 5%, 10%, 15%, and 20% Reindeer Lichen formulations had no notable differences after testing. All formulations were able to be sprayed before and after stability testing. All spray formulations were considered to be stability passing.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A lichen extract-based formulation comprising:
a mixture of reindeer lichen extract (Cladonia rangiferina),
lung lichen extract (Lobaria pulmonaria), and
witch's hair lichen extract (Alectoria sarmentosa).

2. The formulation of claim 1, wherein the reindeer lichen extract is an extract of a whole Cladonia rangiferina plant at a ratio of 10:1, 20:1, 30:1, 40:1 or combinations thereof.

3. The formulation of claim 1, wherein the lung lichen extract is an extract of a whole Lobaria pulmonaria plant at a ratio of 10:1, 20:1, 30:1, 40:1 or combinations thereof.

4. The formulation of claim 1, wherein the witch's hair lichen extract is an extract of a whole Alectoria sarmentosa plant at a ratio of 10:1, 20:1, 30:1, 40:1 or combinations thereof.

5. The formulation of claim 1, wherein the formulation is in a powder form.

6. The formulation of claim 1, further comprising:
protein; fat; fiber; vitamins; minerals; a preservative and an emulsifier, in the form of a protein block, wherein the mixture comprises from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

7. The formulation of claim 6, wherein the reindeer lichen extract is an extract of a whole Cladonia rangiferina plant at a ratio of 10:1, 20:1, 30:1, 40:1 or combinations thereof.

8. The formulation of claim 6, wherein the lung lichen extract is an extract of a whole Lobaria pulmonaria plant at a ratio of 10:1, 20:1, 30:1, 40:1 or combinations thereof.

9. The formulation of claim 6, wherein the witch's hair lichen extract is an extract of a whole Alectoria sarmentosa plant at a ratio of 10:1, 20:1, 30:1, 40:1 or combinations thereof.

10. The formulation of claim 1, further comprising:
protein; fat; fiber; vitamins; minerals; a preservative and an emulsifier, in the form of a feed supplement, wherein the mixture comprises from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

11. The formulation of claim 10, wherein the reindeer lichen extract is an extract of a whole Cladonia rangiferina plant at a ratio of 10:1, 20:1, 30:1, 40:1 or combinations thereof.

12. The formulation of claim 10, wherein the lung lichen extract is an extract of a whole Lobaria pulmonaria plant at a ratio of 10:1, 20:1, 30:1, 40:1 or combinations thereof.

13. The formulation of claim 10, wherein the witch's hair lichen extract is an extract of a whole Alectoria sarmentosa plant at a ratio of 10:1, 20:1, 30:1, 40:1 or combinations thereof.

14. The formulation of claim 1, further comprising: a solution comprising water.

15. The formulation of claim 14, wherein the reindeer lichen extract is an extract of a whole Cladonia rangiferina plant at a ratio of 10:1, 20:1, 30:1, 40:1 or combinations thereof.

16. The formulation of claim 14, wherein the lung lichen extract is an extract of a whole Lobaria pulmonaria plant at a ratio of 10:1, 20:1, 30:1, 40:1 or combinations thereof.

17. The formulation of claim 14, wherein the witch's hair lichen extract is an extract of a whole Alectoria sarmentosa plant at a ratio of 10:1, 20:1, 30:1, 40:1 or combinations thereof.

18. A lichen extract-based spray cleaner formulation, comprising:
an ingredient comprising at least one of reindeer lichen extract (Cladonia rangiferina) and lung lichen extract (Lobaria pulmonaria);
an enzyme stabilizer;
preservative;
antimicrobial active; and
a carrier solvent.

19. The spray formulation of claim 18, wherein the carrier solvent is water.

20. The spray formulation of claim 18, wherein the ingredient is present in an amount of from 5% to 40%.

21. A method, comprising:
administering to cervids a composition comprising a mixture of reindeer lichen extract (Cladonia rangiferina), lung lichen extract (Lobaria pulmonaria), and witch's hair lichen extract (Alectoria sarmentosa).

22. The method of claim 21, wherein the composition further comprises:
protein; fat; fiber; vitamins; minerals; a preservative and an emulsifier, in the form of a protein block, wherein the mixture comprises from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

23. The method of claim 21, wherein the composition further comprises:
protein; fat; fiber; vitamins; minerals; a preservative and an emulsifier, in the form of a feed supplement, wherein the mixture comprises from 1% to 40% by weight reindeer lichen extract (Cladonia rangiferina), from 1% to 40% by weight lung lichen extract (Lobaria pulmonaria), and from 5% to 45% by weight witch's hair lichen extract (Alectoria sarmentosa).

24. The method of claim 21, wherein the composition further comprises a solution comprising water.
